(19) United States
(12) Patent Application Publication
(10) Pub. No.: US 2006/0199189 A1
(43) Pub. Date: Sep. 7, 2006

(54) Low-dose, sequenced, individualized chemotherapy dosing method

(76) Inventor: Sherry A. Bradford, Grand Island, NY (US)

Correspondence Address: WALTER W. DUFT;LAW OFFICES OF WALTER W. DUFT, 8616 MAIN ST, SUITE 2, WILLIAMSVILLE, NY, 14221, US

(21) Appl. No.: 11/073,925

(22) Filed: Mar. 7, 2005

(51) Int. Cl. C12Q 1/68; A61K 9/22

(52) U.S. Cl. 435/6; 604/890.1

(57) ABSTRACT

A low-dose, sequenced, individualized chemotherapy dosing method wherein an identification is made of biomorphomolecular markers associated with a patient tumor to be treated, and a percentage of tumor cells expressing each of the biomorphomolecular markers is determined. A set of drugs is selected that are adapted to target the biomorphomolecular markers. A percent of peak plasma concentration is determined for each drug based on the percentage of cells respectively expressing each biomorphomolecular marker. The drugs are administered according to drug dosages based on the respective concentrations. The method further includes selecting a drug treatment period, selecting a drug repetition cycle within the drug treatment period, determining a drug dosage for each drug based on its concentration and the number of repetition cycles, determining a drug sequence to be given during each cycle, and determining a dosing time for each drug.

### BACKGROUND OF THE INVENTION

1. Field of the Invention

The present invention relates to the treatment of cancer. More particularly, the invention concerns the determination and administration of a course of chemotherapy treatment following evaluation of patient cancer testing.

2. Description of Prior Art

By way of background, cancer patients undergoing chemotherapy are generally treated based on diagnoses made by physicians using standard and generic protocols, with the type of protocol being largely determined according to the tumor's generic histologically-determined stage, morphological characteristics (e.g., cell size and shape), and the individual clinician's experience and preference. Conventional chemotherapy protocols are implemented using bolus or intravenous infusion delivery of a selected drug or combination of drugs. The drugs are typically administered at peak plasma concentrations on a multi-weekly basis. More recently, the concept of dose density has given rise to proposals for more frequent (e.g., weekly) administration of lower doses in order to improve toxicity profiles by avoiding the high-peak drug concentrations associated with multiweekly regimens. However, a continuing need exists for further improvements in the administration of cancer treatment chemotherapy in order to increase its effectiveness as an anti-cancer therapy regimen and minimize adverse side effects.

### SUMMARY OF THE INVENTION

The foregoing objects are achieved and an advance in the art is provided by a low-dose, sequenced, individualized chemotherapy dosing method that is characterized by drug administration sequences measured in hours rather than weeks, and which uses less than peak plasma concentrations of drugs that are selected according to the requirements of individual patients rather than on tumor type and stage, as is presently done. According to exemplary aspects of the invention, an identification is made of ubiquitously expressed biomorphomolecular markers that are associated with the specific patient's tumor that is to be treated. The percentage of tumor cells expressing each of the biomorphomolecular markers is determined, and a set of drugs is selected that are specifcally adapted to target each biomorphomolecular marker. A percent of peak plasma concentration is then determined for each of the drugs based on the respective percentage of cells expressing each of the biomorphomolecular markers, and the drugs are administered according to drug dosages based on the percent of peak plasma concentration.

The inventive method according to additional exemplary aspects further includes selecting a drug treatment period, selecting a drug repetition cycle within the drug treatment period, determining a drug dosage for each of the drugs based on the percent of peak plasma concentration for each of the drugs and the number of repetition cycles that will occur during the drug treatment period, determining a sequence of the drugs to be given during each of the drug repeition cycles, and determining a dosing time for each of the drugs. The drugs are then administered according to the drug dosages, the sequence of drugs and the dosing times during each of the drug repetition cycles until the drug treatment period has expired. The drug treatment period can be selected to achieve a desired log-kill, with a period of approximately one seven-day week being typical. The drug repetition cycle can be selected based on consideration of tumor cell cycle, with a cycle of approximately one twenty-four hour day being typical. The drug seqeunce can be selected based on consideration of drug synergy, additivity and inhibition. The drug sequence can also be based on the percent of peak plasma concentration for each of the drugs, as by adminsitering the drugs in decreasing order of concentration. The drug sequence can be varied for each of the repetition cycles. For example, a drug that was first to be administered in a repetition cycle n-1 could be the last to be administered in next repetition cycle n. The dosing time can be determined by the repetition cycle divided by the number of drugs to be administered, and taking into account rest periods allocated for the repeition, if any. The drugs can be sequenced by way of infusion pumping with automated control to selectively adminster each of the drugs at different times. Should a specific drug have to administered orally, due to its mechanism of action, rather than by IV route, this will be initiated during the cycle where deemed appropriate.

### DETAILED DESCRIPTION OF EXEMPLARY EMBODIMENTS

The present invention can be implemented in conjunction with the cancer diagnostic techniques disclosed in commonly-owned U.S. patent application Ser. No. 10/340,858, filed on Jan. 10, 2003 (the '858 application), the contents of which are incorporated herein by this reference in their entirety. As taught by the '858 application, a solid tumor is a heterogeneous composite of mutated cells and may also contain normal cells that have been caught-up and entangled in the mass. This fact predicates that the tumor is of necessity, a composite of multiple biomorphomolecular markers (BMMMs) that are indicative of the specific tumor's pathological nature, including the type and stage of the malignancy. The BMMMs will encompass growth-favorable and maintenance-favorable parameters such as indicators/markers of cell cycle phase, signal transduction pathways, nuclear activation, mRNA activation, extracellular and cytoplasmic growth markers, vascularization, apoptosis, multi-drug resistance proteins, inflammatory proteins, adhesion proteins, and basement membrane escape. Generally, however, the markers normally associated with a tumor, referred to as “Tumor Markers” per se, are not themselves the entities that recognize and bind to specific growth and maintenance promoting receptors, and cannot prevent their growth and maintenance. This mandates that in order for chemotherapy to be efficacious, a multiple of tumor enhancing/promoting BMMMs must be considered and tabulated in totality.

The '858 application instructs that one drug generally cannot target a tumor composed of a multiple of entities. Moreover, one person's staged tumor type cannot be exact in another human of the same staged tumor type. Therefore, one drug will usually not be efficacious for every human of that same staged tumor type. Furthermore, tumor “sameness” does not translate into treatment “sameness” especially because there is biochemical and genetic “disparity” between “same” tumors on particular and diverse individuals.

As described by way of background above, chemotherapy drugs are typically administered at peak plasma concentrations on a multi-weekly basis, or at lower dose densities and somewhat shorter time intervals, such as once per week. Adminstration is performed by way of bolusing over a very short time period. It is hypothesized that when a drug is conventionally administered in this fashion at a peak or near plasma concentration that a number of consequences can ensue:
1) The tumor will not understand the “death” signal when a drug is given in a large amount at one time;
2) The tumor does not “see” the drug, and the patient eliminates most of the drug prior to eliciting its efficacy; and
3) The tumor is given an opportunity to amass multi-drug resistance proteins.
   These parameters result in tumor “escape” from the “kill” therapy, whereby the tumor achieves “life” and hence continued fortification and growth. It is additionally hypothesized that the conventional combining of multiple of drugs (drug “cocktail”) can lead to inhibition, additivity and/or synergy of one or more of the drugs.

The present invention contemplates and reflects these effects and attempts to minimize inhibition while promoting additivity and synergy. This rational purports the administration of lower dosages instead of the gold-standard exploitation of peak plasma or near peak plasma concentrations (the invention further takes into consideration the biological circadian clock and known gender metabolic differences). It is believed that this approach can also prevent hibernation or dormancy of tumor cells that may have escaped drug exposure, thereby minimizing and perhaps eliminating the risk of relapse, recurrence or metastasis. There should also be decrease, if not total eradication, of the overt toxic side effects typically associated with chemotherapy regimen, with consequent restoration of quality and quantity of life.

Based on the teachings of the '858 application, the present invention contemplates that an ELISA reader or comparable device will be used to evaluate an assay containing a sample of the patient's bodily fluid and a panel of antibodies sensitive to specific BMMMs. The up-regulation or down-regulation of one or more BMMMs designed to be identified by the panel may be indicative of a cancerous condition and will indicate a combination of drugs that can be used for effective treatment. Once the desired drug combination of drugs is selected, a determination is then made of the most efficacious regimen for individualizing the drug therapy within a selected time frame for targeting first-line chemotherapies. This will typically be based on a twenty-four hour repetition cycle and will target the cell cycle of growing tumor cells. In particular, all drugs to be administered will be infused in sequence once every twenty-four hours over a selected treatment period, such as a seven-day week. Other repetition cycles, such as twelve hours, thirty-six hours, etc., could also be used. The goal is to preferably capture the cell cycle of the tumor so that the drugs being administered are given the ability to target cells within the growing/expansion phases (s-phase fraction and higher), cells within the Go phase and undergoing stimulation, cells that are not actively engaged in proliferation but are at a potential for active deployment (i.e., chemically, radiologically or surgically assaulted and/or sloughed off during those procedures), or any fibroblasts, EC, cuboidal columnar cells, etc., that comprise the surrounding tissues.

The treatment period is selected to provide a sufficient number of repetition cycles to ensure that the drugs are able to repeatedly assault the tumor, with each cycle producing some fractional cell kill based on the concept of fractional or log-kill. This hypothesis holds that a given drug concentration applied for a defined time period will kill a fraction of the cell population, independent of the absolute number of cells. Thus, each repetition cycle kills a specific fraction of the remaining cells and the goal is thus to implement a sufficient number of repetition cycles to reduce the absolute number of remaining cells to zero, or less than 1. For example, if there is a tumor burden of 10¹¹, obtaining a cell kill to zero or less than 1 will require six repetition cycles (10¹¹×0.01⁶=0.1). A seven day treatment period should be satisfactory for most chemotherapy scenarios. Other treatment periods, such as five days, ten days, etc., could also be used.

Following determination of the repetition cycle and treatment period, the amount of BMMM expression as determined by the patient's assay results is used to determine the dosage for each drug to be infused. That is, once algorithms have established the estimated number of cells within the entire tumor cell mass, a percent expression of specific BMMMs will be calculated. Based on the number of cells expressing each BMMM, a proportionate amount of drug will be administered rather than a peak plasma (100%) concentration. The proportionate concentration of drug to be administered is given as a percent of peak plasma or “PP.” The percent of peak plasma of a drug to be administered corresponds to the percent of BMMM expression, and preferably either equal to that percentage or slightly above (e.g. +5%) to provide a margin of safety. For example, given a determination that a tumor is expressing BMMMs A, B, C, D, E and F at the various percentages given below in Table 1, drug concentrations that correspond to these percentages will be administered, as follows:

TABLE 1
Drug PP Concentration Based On BMMM Expression

| BMMM | PERCENT EXPRESSION | DRUG CONCENTRATION |
|---|---|---|
| TYPE | (% TUMOR CELLS) | (% PEAK PLASMA) |
| BMMM A | 75% | 80% |
| BMMM B | 50% | 55% |
| BMMM C | 25% | 30% |
| BMMM D | 75% | 80% |
| BMMM E | 10% | 15% |
| BMMM F | 100% | 100% |

A drug's peak plasma concentration can be defined as the drug's recommended dosage per manufacture specifications. For example, the indicated dosage for the drug Cisplatin (sold by Novation, LLC) for treating testicular tumors is 20 mg/m² (patient body surface area) daily for five days. The peak plasma concentration for Cisplatin is thus taken to be 20 mg/m²×5=100 mg/m². The percent of peak plasma concentration for Cisplatin will be some number that is less than the peak plasma concentration. For example, if Cisplatin is the drug that targets BMMM A above, the percent of peak plasma dosage would be 0.75×100 mg/m²=75 mg/m². In order to determine the actual drug dosage to be given during a single repetition cycle, the calculated percent of peak plasma concentration is divided by the number of repetition cycles within the treatment period. For example, if the treatment period is seven days and the repetition cycle is twenty-four hours, there will be seven repetition cycles over the course of the treatment. Continuing the example above where Cisplatin is the drug that targets BMMM A, the dosage per repetition cycle of this drug would be 75 mg/m²÷7=10.71 mg/m²/24 hr period(day)/for 7 days.

Once all drug dosages are calculated, the drugs can be sequenced with intravenous (IV) drip port infusion using a pre-programmed locked pump. Intraperitoneal or intravenous (systemic) delivery may be used, or both. Preferably, the patient will have a port installed in which to attach a tube that communicates with the outlet side of the pump. The inlet side of the pump will connect to individual drug reservoirs, each of which contains the correct dosage of one of the drugs. Valves operating under automated control will regulate the flow of drugs from the individual drug reservoirs. The valves and automated control functionality may be incorporated in the pump, or they may be implemented separately, such as in a manifold that connects each of the drug reservoirs to the pump inlet. During drug sequencing, the pump will drip a drug from its reservoir to the port attached to the patient. When it is determined that one drug has been dispensed and another is ready for delivery, the valve for the first drug's reservoir will close and the valve for the second drug resevoir will open. The process will repeat until all drugs are delivered. Note that this procedure will allow the pateint to be totally ambulatory and thus home in a healing-promoting environment. However, should such a pump unit not be available, a visiting nurse scenario or hospital setting may be initiated.

The dosing time for each drug can be calculated by dividing the length of the repetition cycle by the number of drugs to be administered, e.g., 24 hours÷6 drugs=4 hours each. Alternatively, the dosing time does not have to be the same for each drug and could be calculated in some other way, such as by weighting the drip time according to each drug's peak plasma concentration. In addition, there is no requirement that drugs be administered throughout the repetition cycle, and provision can be made for one or more “rest” periods. For example, if a twenty-four hour repetition cycle is used, the drugs could be delivered for twelve hours, followed by twelve hours “off,” or eighteen hours “on,” followed by six hours “off.” A further variation would be to allocate a rest period between each drug. For example, given a twenty-four hour repetition cycle and six drugs, each drug could be administered for two hours and a two hour rest period could be observed between each drug.

For the first repetition cycle, the actual sequence of drug infusion can be deteremined according to parameters such as the time that one drug is to follow another in realizing an additive or synergistic effect versus actually lessening the effect (antagonism) of any one of the drugs for a given patient. Consideration should be given to the various mechanisms by which each drug works based on parameters such as cell cycle, DNA, multiple drug resistance, signal transduction pathways, etc. Thus, the therapist would not administer two drugs in sucession that target the same signal transduction pathway, and would instead deliver the drugs so that one drug is delivered further upstream or downstream of the other during the repetition cycle.

In some cases, the order of drug delivery may be selected to correspond to the percent of BMMM expression in the tumor mass. Thus, the drug targeting the BMMM with the highest expression can be first, following by the remaining drugs in descending order of BMMM expression down to the drug targeting the least expressed BMMM. According to Table 1 above, BMMM F has the highest expression at 100%, followed by BMMM A and BMMM D at 75%, BMMM B at 50%, BMMM C at 25%, and BMMM E at 10%. The drug targeting BMMM F would thus be infused first, the drugs targeting BMMMs A and D would be infused second and third (or visa versa), the drug targeting BMMM B would be infused fourth, the drug targeting BMMM C would be infused fifth, and the drug targeting BMMM E would be infused sixth.

Following each repetition cycle in which all drugs are administered, a new repetition cycle begins and the administration of all drugs is repeated, but preferably with the drug order altered, such as being staggered by one. Using the staggered approach, a drug that was the first to be administered in the preceding repetition cycle will be the last to be administered in the current repetition cycle. All other drugs will move up one position in the delivery sequence. Thus, the second drug in the preceding repetition cycle will become the first drug to be administered in the current repetition cycle, and so on.

Table 2 below illustrates an exemplary chemotherapy dosing program for the BMMMs A-F based on the foregoing strategies, with each repetition cycle being a twenty-four hour day and each drug being infused for an equal amount of time during each day:

TABLE 2
Drug Sequence

| Day/Drug | Day 1 | Day 2 | Day 3 | Day 4 | Day 5 | Day 6 | Day 7 |
|---|---|---|---|---|---|---|---|
| Drug 1 - | 1 | 2 | 3 | 4 | 5 | 6 | 1 |
| Targeting | Drip for | Turn off | Turn off | Turn off | Turn off | Turn off | Turn off |
| BMMM F | 4 hours | # 6 - | # 1 - Drip | # 2 - | # 3 - Drip | # 4 - | # 5 - |
| | | Drip in | in #3 for | Drip in | in #5 for | Drip in | Drip in |
| | | #2 for 4 | 4 hours | #4 for 4 | 4 hours | #6 for 4 | #1 for 4 |
| | | hours | | hours | | hours | hours |
| Drug 2 | 2 | 3 | 4 | 5 | 6 | 1 | 2 |
| Targeting | Turn off | Turn off | Turn off | Turn off | Turn off | Turn off | Turn off |
| BMMM A | # 1 - | # 2 - | # 3 - Drip | # 4 - | # 5 - Drip | # 6 - Drip | # 1 - |
| | Drip in | Drip in | in #4 for | Drip in | in #6 for | in #1 for | Drip in |
| | #2 for 4 | #2 for 4 | 4 hours | #5 for 4 | 4 hours | 4 hours | #2 for 4 |
| | hours | hours | | hours | | | hours |
| Drug 3 | 3 | 4 | 5 | 6 | 1 | 2 | 3 |
| Targeting | Turn off | Turn off | Turn off | Turn off | Turn off | Turn off | Turn off |
| BMMM D | # 2 - | # 3 - | # 4 - Drip | # 5 - | # 6 - Drip | # 1 - Drip | # 2 - Drip |
| | Drip in | Drip in | in #5 for | Drip in | in #1 for | in #2 for | in #3 for |
| | #3 for 4 | #4 for 4 | 4 hours | #6 for 4 | 4 hours | 4 hours | 4 hours |
| | hours | hours | | hours | | | |
| Drug 4 | 4 | 5 | 6 | 1 | 2 | 3 | 4 |
| Targeting | Turn off | Turn off | Turn off | Turn off | Turn off | Turn off | Turn off |
| BMMM B | # 3 - | # 4 - | # 5 - Drip | # 6 - | # 1 - Drip | # 2 - Drip | # 3 - Drip |
| | Drip in | Drip in | in #6 for | Drip in | in #2 for | in #3 for | in #4 for |
| | #4 for 4 | #5 for 4 | 4 hours | #1 for 4 | 4 hours | 4 hours | 4 hours |
| | hours | hours | | hours | | | |
| Drug 5 | 5 | 6 | 1 | 2 | 3 | 4 | 5 |
| Targeting | Turn off | Turn off | Turn off | Turn off | Turn off | Turn off | Turn off |
| BMMM C | # 4 - | #5 - Drip | # 6 - Drip | # 1 - | # 2 - Drip | # 3 - Drip | # 4 - Drip |
| | Drip in | in #6 for | in #1 for | Drip in | in #3 for | in #4 for | in #5 for |
| | #5 for 4 | 4 hours | 4 hours | #2 for 4 | 4 hours | 4 hours | 4 hours |
| | hours | | | hours | | | |
| Drug 6 | 6 | 1 | 2 | 3 | 4 | 5 | 6 |
| Targeting | Turn off | Turn off | Turn off | Turn off | Turn off | Turn off | Turn off |
| BMMM E | # 5 - | # 6 - | # 1 - Drip | # 2 - | # 3 - | # 4 - Drip | # 5 - Drip |
| | Drip in | Drip in | in #2 for | Drip in | Drip in | in #5 for | in #6 for |
| | #6 for 4 | #1 for 4 | 4 hours | #3 for 4 | #4 for 4 | 4 hours | 4 hours |
| | hours | hours | | hours | hours | | |

The disclosed chemotherapy dosing program may include various constraints and actions in order to ensure patient safety. For example, before recommending a specific drug and before administration, a check can be made to see if the patient is on an anticonvulsive drug. If so, the drug will be non-effective, regardless of its chemical configuration. If possible, it is preferred that preservative-free drugs be used. If not, a reconstituted drug can be run over an absorbent column. A check can also be made to see if the parent drug is not the active moiety or if the drug needs to be metabolized. If it needs to be metabolized, the active metabolite can be acquired. There is also the possibility of administering co-factors, such as vitamins, herbs or other entities, that would facilitate drug absorption by tumor cells. These may be given during a “rest” interval between treatment periods.

It will be appreciated that there are a number of advantages stemming from a chemotherapy dosing program, as disclosed above, that does not automatically administer peak plasma dosing concentrations. For example, individualized targeted chemotherapy can be delivered in an efficacious manner, with drugs being selected based on individualized patient requirements and drug delivery being based on specific cell cycle parameters. Drug synergies and additivities, as well as minimization of inhibition, can also be taken into account.

The dosage of chemotherapy is normally determined on the basis of the body surface area of the patient, which factors in height and weight. This method has proven to be insufficient when it comes to differences among patients in the amount of chemotherapy in the blood. Some patients receive overdoses with severe side-effects as a result, while others receive under-dosed regimens that risk leaving the tumor insufficiently treated. Because the disclosed dosing program uses decreased concentrations of drugs, the probability of toxicity in the patient population is reduced. The net result is to engender the patient less sick (toxic) or not sick at all. Low drug dosages can also obviate the need to give antihistamines, epinephrine, corticosteroids or “anti-allergic” hypersensitivity medication by lowering the risk of side effects such as allergic reaction through the use of repeated low doses rather than fewer large doses. These drugs effectively absorb excess drugs, thereby negating their effectiveness. Moreover, they mobilize white blood cells and may trigger and initiate an immune response that could effectively attack the drug(s) and negate their efficacy.

Drug elimination should also be reduced as a result of using lower drug dosages applied over longer time period, with lowered impairment of renal function ascites and pleural effusion. The tumor will be subjected to more efficient drug exposure because the time that the drug is able to act on the tumor before being metabolized and eliminated by the body will be increased. Chemotherapy can also be administered to hepatic and renal compromised patients. It may also be possible to eliminate the need to administer pretreatment hydration regimens. By administering low concentrations of a combination of drugs in sequence, it is known that combination alone decreases the onset of alopecia (83% of patients as opposed to 100% without combining drugs) as well as other adverse reactions such as myleosuppression, hemorrhagic cystitis, other urologic toxicities, CNS side effects and nausea. By decreasing the amount of drugs given, there is also more time for the host immune system to fend off the invaders by itself. The result will be to thwart low red blood cell counts, low white blood cell counts, low platelet counts, an apathetic and indolent immune system, elicitation of allergic reactions (especially due to amount of drug rather than to the drug moiety itself), the need for drug rescues, the loss of hair (aesthetically appealing), kidney/liver damage, nervous system damage, and pain.

The conventional practice of giving peak plasma drug concentrations when not needed can lead to multidrug resistant expression. This practice also requires more patient monitoring and hence the patient will not usually be ambulatory. Administering peak plasma concentrations of drugs can also make cells senescent instead of killing them. Some chemotherapy drugs that induce senescence of tumor cells cause the expression of the senescent cell-derived inhibitor (sdi)-1 protein (p21 product). This gene then turns on neighboring cells. The senescent cells not only activate genes that inhibit cell division, they also activate genes that stimulate, rather than prevent the growth of neighboring cells, as well as the p21 gene. The p21 gene activates a host of genes linked to numerous diseases. Such genes have been shown to be activated in senescent colon cancer cells. A lowered dosing rational may prevent p21 expression.

By infusing chemotherapy drugs drip wise over a period of time at appropriate dosages based on the percentage of corresponding BMMMs, there should be an eradication of tumor mass. Moreover, tumor cells and tumor “decoy” antigens that slough off and become soluble (circulate in the blood) can be targeted. These soluble antigens cannot merely be discarded by the body via the immune system. They will remain dormant somewhere in the body but can be neutralized when they activate if chemotherapy drugs are present on continuous basis, as provided for herein.

The disclosed dosing program further enables the administration of preservative-containing drugs. Because preservatives seem to be the allergen rather than the drug moiety itself, exposure to a lesser amount would be less likely to elicit an immune response from the patient. It should also be possible to avoid bringing on Fanconi syndrome renal rickets, renal tubular acidosis and other conditions brought on by cytotoxic drug administration.

Accordingly, a low-dose, sequenced, individualized chemotherapy dosing method has been disclosed. While various embodiments and features of the invention have been described, it should be apparent that many variations and alternative embodiments and features could be implemented in accordance with the invention. It is understood, therefore, that the invention is not to be in any way limited except in accordance with the spirit of the appended claims and their equivalents.

What is claimed is:

1. A low-dose, sequence dripped, individualized chemotherapy dosing method, comprising:
identifying biomorphomolecular markers associated with patient tumor to be treated;
determining a percentage of tumor cells expressing each of said biomorphomolecular markers;
selecting a set of drugs adapted to target said biomorphomolecular markers;
determining a percent of peak plasma concentration for each of said drugs based on said percentage of cells respectively expressing said biomorphomolecular markers; and
administering said drugs according to drug dosages based on said percent of peak plasma concentration.

2. A method in accordance with claim 1 further including seleting a drug treatment period over which said drugs are given.

3. A method in accordance with claim 2 further including selecting a drug repetition cycle within said drug treatment period.

4. A method in accordance with claim 3 wherein said drug dosages are based on said percent of peak plasma concentration for each of said drugs and the number of said repetition cycles that will occur during said drug treatment period.

5. A method in accordance with claim 4 further including selecting a sequence of said drugs to be given during each of said drug repeition cycles.

6. A method in accordance with claim 5 further including selecting a dosing time for each of said drugs to be given during said sequences.

7. A method in accordance with claim 6 wherein said drugs are administered according to said drug dosages, said sequence of drugs and said dosing times during each of said drug repetition cycles until said drug treatment period has expired.

8. A method in accordance with claim 2 wherein said drug treatment period is selected to achieve a desired log-kill.

9. A method in accordance with claim 8 wherein said drug treatement period is approximately one seven-day week.

10. A method in accordance with claim 3 wherein said drug repetition cycle is selected based on consideration of tumor cell cycle.

11. A method in accordance with claim 10 wherein said drug repetition cycle is approximately one twenty-four hour day.

12. A method in accordance with claim 5 said drug seqeunce is selected based on consideration of drug synergy, additivity and inhibition.

13. A method in accordance with claim 5 wherein said drug sequence is selected based on said percent of peak plasma concentration for each of said drugs.

14. A method in accordance with claim 5 wherein said drug sequence is varied for each of said repetition cycles.

15. A method in accordance with claim 5 wherein said drug sequence is varied for each of said repetition cycles so that a drug that was first to be administered in a repetition cycle n-1 is last to be administered in next repetition cycle n.

16. A method in accordance with claim 6 wherein said dosing time is determined by said repetition cycle divided by the number of said drugs to be administered.

17. A method in accordance with claim 16 wherein said dosing time is determined based said repetition cycle including one or more rest periods.

18. A method in accordance with claim 1 wherein said drugs are sequenced by way of infusion pumping with automated control to selectively adminster each of said drugs at different times.

19. A low-dose, sequence dripped, individualized chemotherapy dosing method, comprising:
identifying biomorphomolecular markers associated with patient tumor to be treated;
determining a percentage of tumor cells expressing each of said biomorphomolecular markers;
selecting a set of drugs adapted to target said biomorphomolecular markers;
determining a percent of peak plasma concentration for each of said drugs based on said percentage of cells respectively expressing said biomorphomolecular markers;
selecting a drug treatment period;
selecting a drug repetition cycle within said drug treatment period;
determining a drug dosage for each of said drugs based on said percent of peak plasma concentration for each of said drugs and the number of said repetition cycles that will occur during said drug treatment period;
determining a sequence of said drugs to be given during each of said drug repeition cycles;
determining a dosing time for each of said drugs; and
administering said drugs according to said drug dosages, said sequence of drugs, and said dosing times during each of said drug repetition cycles until said drug treatment period has expired.

20. A low-dose, sequence dripped, individualized chemotherapy dosing method, comprising:
identifying biomorphomolecular markers associated with patient tumor to be treated;
determining a percentage of tumor cells expressing each of said biomorphomolecular markers;
selecting a set of drugs adapted to target said biomorphomolecular markers;
determining a percent of peak plasma concentration for each of said drugs based on said percentage of cells respectively expressing said biomorphomolecular markers;
selecting a drug treatment period;
selecting a drug repetition cycle within said drug treatment period;
determining a drug dosage for each of said drugs based on said percent of peak plasma concentration for each of said drugs and the number of said repetition cycles that will occur during said drug treatment period;
determining a sequence of said drugs to be given during each of said drug repeition cycles;
determining a dosing time for each of said drugs; and
administering said drugs according to said drug dosages, said sequence of drugs and said dosing times during each of said drug repetition cycles until said drug treatment period has expired;
said drug treatment period being selected to achieve a desired log-kill and being approximately one seven-day week;
said drug repetition cycle being selected based on consideration of tumor cell cycle and being approximately one twenty-four hour day;
said drug seqeunce being selected based on consideration of drug synergy, additivity and inhibition or based on said percent of peak plasma concentration for each of said drugs;
said drug sequence being varied for each of said repetition cycles so that a drug that was first to be administered in a repetition cycle n-1 is last to be administered in next repetition cycle n;
said dosing time being determined by said repetition cycle divided by the number of said drugs to be administered and taking into account rest periods allocated for said repeition, if any; and
said drugs being sequenced by way of infusion pumping with automated control to selectively adminster each of said drugs at different times.
